(19) United States
(12) Patent Application Publication
(10) Pub. No.: US 2008/0064675 A1
(43) Pub. Date: Mar. 13, 2008

(54) Use of Aromatic flavoring agents with high melting point as solubilizing agents

(76) Inventor: Henry Jun Suzuki, Sao Paulo (BR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/889,764

(22) Filed: Aug. 16, 2007

Related U.S. Application Data
(63) Continuation-in-part of application No. PCT/BR06/00024, filed on Feb. 16, 2006.

(30) Foreign Application Priority Data
Feb. 16, 2005 (BR) PI 0500520-5

(51) Int. Cl. A61K 47/00; A61K 31/4535; A61K 47/12; A61K 47/44; A61K 31/585

(52) U.S. Cl. 514/173; 514/324; 514/618; 514/772; 514/783; 514/784

(57) ABSTRACT

The present invention relates to the use of aromatic flavoring compounds having a high melting point as solubilizing agents or crystallization inhibitor agents of insoluble or poorly-water soluble biologically active ingredients in pharmaceutical and agrochemical compositions. Additionally, the present invention also relates to pharmaceutical compositions containing aromatic flavoring compounds with a melting point higher than 20° C. as solubilizing agents or crystallization inhibitor agent as well as a process for solubilizing or inhibiting crystallization of at least one insoluble or poorly-water soluble biologically active ingredient in a pharmaceutical or agrochemical composition.

This application is a continuation-in-part of PCT/BR2006/000024, filed Feb. 16, 2006, the disclosure of which is incorporated by reference herein.

The present invention relates to the use of at least one aromatic flavoring compound with a high melting point as a solubilizer or crystallization inhibitor of an insoluble or poorly-water soluble active ingredient in a pharmaceutical composition. The present invention also relates to a pharmaceutical composition or agrochemical composition comprising at least one aromatic flavoring compound with a high melting point as a solubilizing agent or a crystallization inhibitor. The present invention further relates to a process for solubilizing or inhibiting crystallization of at least one insoluble or poorly-water soluble biologically active ingredient in a pharmaceutical or agrochemical composition, the process comprising bringing at least one aromatic flavoring compound with a high melting point into contact with the at least one biologically active ingredient in the pharmaceutical or agrochemical composition.

One of the main problems when developing new pharmaceutical products is the low solubility in water presented by several active ingredients, which brings about the necessity for lower particle sizes as well as use of specific excipients to increase the adsorption and absorption rate of such ingredients.

Some examples of excipients used for low water soluble active ingredients solubilization include pH modifiers, water soluble organic solvents, surfactants, water insoluble organic solvents, long and medium chain triglycerides, cyclodextrins, and phospholipids.

Bearing in mind the importance of considering the toxicity and safety of new excipients used for pharmaceutical products, it is not surprising that a variety of liquid compounds found in food products have been studied as solvents in pharmaceutical products. Examples of such liquid compounds include vegetable edible oils (for example, soy oil, linseed oil, corn oil, etc.), essential oils (for example, mint oil, clove oil, lemon oil, etc.) or chemically pure compounds, liquid at room temperature (for example, d-alpha-tocopherol, oleic acid, stearic acid, mono and di-glycerides, etc.).

Although such excipients have been used with relative success for many commercial products, there is still an important limitation on formulation of specific active ingredients, for example those active ingredients having strong intermolecular interactions involving aromatic groups, such as modafinil and trans-anetholtrithione, due to limitations on solubilizing capacity or toxicity showed by these products.

In fact, the safety question seems to be especially concerned with the use of vegetable essential oils because of the recognized toxicity of certain agents they contain and because the lower daily dose considered as safe to other components leads to limitations in the use of such compounds “in natura” in pharmaceutical formulations. By way of example, products such as safrole and estragole, found in essential oils of many vegetable species are reported as potential carcinogenic agents.

In order to solve the problems described above, the inventors searched to find compounds which are capable of providing both improved solubility and safety. As a result of this search, the present inventor found that although they are solid at temperatures lower than 20° C. (which presumably would make them poorly attractive to be used as a solvent), aromatic flavoring compounds, such as vanillin and trans-anethole, were useful for solubilizing and inhibiting crystallization of insoluble or poorly water soluble compounds, for example compounds which present aromatic groups inside their chemical structure. Some examples of insoluble or poorly water soluble compounds that may be solubilized or that the crystallization may be inhibited according to the present invention are modafinil, drospirenone, eplerenone, raloxifene and anetholtrithione. In particular, some examples of insoluble or poorly water soluble compounds according to the present invention are compounds that have aromatic groups inside their chemical structure, such as modafinil and anetholtrithione. It should be noted, however, that the invention may be used to solubilize and/or inhibit crystallization of any insoluble or poorly soluble molecule such as pharmaceutical drugs, pesticides, herbicides, proteins, amino acids, vitamins, antibiotics, and other similar substances.

### DETAILED DESCRIPTION OF THE INVENTION

In one embodiment, the present invention comprises the use of at least one aromatic flavoring compound having a high melting point, for example, a melting point higher than 20° C., as a solubilizing agent and a crystallization inhibitor agent of insoluble or poorly-water soluble biologically active substances. In another embodiment, the present invention comprises a pharmaceutical or agrochemical composition comprising at least one aromatic flavoring compound having a melting point higher than 20° C., as a solubilizing agent or crystallizing inhibitor agent, wherein the amount of the at least one aromatic flavoring compound in the composition is higher than one tenth of the mass of the active ingredient. In another embodiment, the present invention relates to a process for solubilizing or inhibiting crystallization of at least one insoluble or poorly-water soluble biologically active ingredient in a pharmaceutical or agrochemical composition, the process comprising bringing at least one aromatic flavoring compound having a melting point higher than 20° C. into contact with at least one active ingredient. In yet another embodiment, the present invention relates to the above process, wherein the aromatic flavoring compound is present in an amount higher than 10% by wt relative to the other excipients.

According to the present invention, the expression “aromatic flavoring compound” comprises edible substances commonly used to modify food or medicine flavors, such as those contained in the “Flavouring Agents Database” from Joint FAOIWHO Expert Committee on Food Additives (JECFA), currently accessible through the site http://www.fao.org/ag/agn/jecfa-flav/search.html?lang=en, which contain at least one aromatic ring inside its chemical structure.

In some embodiments of the present invention, the at least one aromatic flavoring compound is a benzilic compound hydroxy- or alcoxy-substituted. In another embodiment of the present invention the, at least one aromatic flavoring compound is a compound in which daily ingestion, commonly accepted as safe, is more than 10 mg. Some examples of an aromatic flavoring compound with a melting point higher than 20° C. comprised by the present invention are trans-anethole(1-metoxy-4-(1E)-1-propenylbenzene), vanillin(4-hydroxy-3-methoxybenzaldehyde), ethylvanillin(4-etoxy-3-methoxybenzaldehyde), coumarin(2H-1-benzopyran-2-one), benzyl benzoate (benzoic anhydride) or mixtures thereof, preferred compounds include trans-anethole and vanillin as well as mixtures thereof.

The aromatic flavoring compounds as comprised by the present invention are substances commonly used to modify the flavor of food compositions or medicines. However, the use of these aromatic flavoring compounds as solubilizing agents or crystallization inhibitor agents, as described herein, is different from how these compounds are commonly used for at least the following reasons: (i) when the aromatic flavoring compounds with a melting point higher than 20° C. in accordance with the present invention are used, there is dissolution of a significant amount of one or more active ingredients as compared with corresponding compositions wherein such aromatic flavoring compounds are not used; (ii) the suppression of use or reduction of the used amount of aromatic flavoring compound with a melting point higher than 20° C. leads to phase separation, precipitation, or crystallization of a significant amount of one or more active ingredients of the composition, which occurs more rapidly as compared with corresponding compositions comprising the aromatic flavoring compounds according to the present invention (a “significant amount” may be the portion of 1% or more, based on the total mass of active ingredient); and (iii) the aromatic flavoring compounds with a melting point higher than 20° C. are used in an amount higher than one tenth of the active ingredient mass to obtain solubilization or crystallization inhibition.

In another embodiment, the present invention comprises a pharmaceutical or agrochemical composition comprising at least one active ingredient significantly dissolved or precipitated on an amorphous form directly in contact with at least one aromatic flavoring compound having a melting point higher than 20° C. Some examples of compositions comprised by the present invention are solutions, emulsions, suspensions, micellae, liposomes, nanoparticles of biodegradable polymers, self-emulsionable compositions, particulated compositions having an adsorbed liquid phase, gels, paste, creams, foams, etc. Other examples of compositions comprised by the present invention are self-emulsifying liquid compositions, for example in the form of micro or nanoemulsions, to increase the absorption rate of active ingredients or even reducing the potential side effects due to direct contact between compositions and the gastric or intestinal mucous membrane in the case of pharmaceutical compositions.

In another embodiment, the present invention relates to a pharmaceutical intermediary composition in which there is solubilization of a significant amount of at least one active ingredient with the help of at least one aromatic flavoring compound with a melting point higher than 20° C. in one or more steps of the process of producing the formulation, even if the finished composition does not have a significant amount of active ingredients in solution. One example is a powder formulation from a drying process of the solution containing at least one aromatic flavoring compound with a melting point higher than 20° C. and one or more active ingredients dissolved with its help.

In yet another embodiment of the invention, the composition comprised by the present invention may further contain at least one additional excipient present in the liquid form at room temperature, for example an aromatic organic solvent, such as benzilic alcohol or ethyl benzoate.

Some additional examples of excipients which may be used in association with the aromatic flavoring compound with a melting point higher than 20° C. are chosen from water soluble organic solvents such as low molecular weight polyethyleneglycol such as polyethyleneglycol 200, polyethyleneglycol 300 and polyethyleneglycol 400, ethanol, propyleneglycol, glycerin, n-methylpyrrolidone, dimethylacetamide, dimethyl sulfoxide, and others; nonionic surfactants such as Cremofor EL, Cremofor RH 40, Cremofor RH 60, d-alpha-tocopherol, polyethyleneglycol succinate 1000, polysorbate 20, polysorbate 80, Solutol H15, sorbitane monooleate, poloxamer 407, Labrafil M-1944CS, Labrafil M-2125CS, Labrasol, Gellucire 44/14, Soften 767, polyethyleneglycol mono- and di-fat esters, and others; water insoluble organic solvents such as linseed oil, corn oil, cottonseed oil, olive oil, peanut oil, mint oil, sunflower oil, soy oil, vegetable hydrogenated oils, medium chain triglycerides of coconut oil, and others; semisolid and liquid organic compounds such as bee's wax, oleic acid, medium chain mono- and di-glycerides and others; and phospholipids such as hydrogenated soy phosphatidylcholine, di-stearoyl-phosphatidylglycerol, L-α-di-myristoilphosphatidyl-choline, L-α-di-myristoilphosphatidyl glycerol, and others; and pH regulators and mixtures thereof. In other embodiments of the present invention, one or more of the additional excipients listed above may be used with the aromatic flavoring composition.

In another embodiment of the present invention, the aromatic flavoring composition comprises at least vanillin and anethol or mixtures thereof.

According to yet another embodiment, the aromatic flavoring compound incorporated into the composition is essentially free of impurities. In this context, the term “essentially free of impurities” refers to raw materials containing less than about 5% wt of synthetic or natural impurities in connection with the total mass, such as less than about 1% of impurities from synthesis or purification.

The present invention is additionally illustrated by the following experimental examples. It should be noted that the invention is not limited by the following description.

### EXAMPLE 1

Use of the trans-anethole as solubilizing agent in compositions of anetholtrithione in ethanol: 2 g of trans-anetholtrithione was weighed inside two 200 mL round bottom flasks. 40 g of ethanol 96° GL was added to one of the flasks and to the other flask a mixture of 20 g ethanol 96° GL and 20 g of trans-anethole was added. Both mixtures were heated at a temperature of 50° C. in a bath of thermal oil for 20 minutes under stirring. There was complete dissolution of the anetholtrithione in the flask containing the mixture of trans-anethole with ethanol, and the formation of crystals inside such a flask was not noted for a period of at least 30 minutes after cooling at room temperature, whereas in the flask containing only ethanol, there was no complete dissolution of the anetholtrithione even by heating.

### EXAMPLE 2

Use of vanillin as inhibiting agent for crystallization in compositions of anetholtrithione in trans-anethole: 2 g of trans-anetholtrithione was weighed inside two 200 mL round bottom flasks. 20 g of trans-anethole was added to one of the flasks and to the other flask a mixture of 20 g of trans-anethole and 2 g of vanillin was added. Both mixtures were heated at 50° C. in a bath of thermal oil for 20 minutes under stirring. There was complete solubilization of the anetholtrithione in both flasks. Formation of crystals was not noticed in the flask containing the mixture of trans-anethole with vanillin, in a period of at least 30 minutes after cooling its content at room temperature, whereas in the flask containing only trans-anethole, the formation of crystals of anetholtrithione was noted as soon as the solution achieved room temperature.

### EXAMPLE 3

### Use of Trans-Anethole and Vanillin in Compositions of Modafinil

200 mg of modafinil were weighted in two test tubes (A and B). 2 mL of benzyl alcohol were added to test tube A and 200 mg of vanillin, 2 mL of anethole and 2 mL of benzyl alcohol were added to test tube B. The test tubes were stirred and heated to 60° C. Further, the tubes were cooled at room temperature. Although modafinil was completely solubilized when both tubes were heated to 60° C., it promptly crystallized when tube A was cooled while no crystals were noted in tube B, 12 hours after cooling.

### EXAMPLE 4

### Use of Anethole and Vanillin in Compositions Having Anetholtrithione

200 mg of anetholtrithione were added to 7 test tubes (A, B, C, D, E, F, and G). 2 mL of benzyl alcohol and 2 mL of benzyl benzoate were added to tubes A and B, respectively. A mixture of 2 mL of anethole and 2 mL of benzyl benzoate was added to tube C. A mixture of 2 mL of anethole and 2 mL of benzyl alcohol was added to tube D. Mixtures of 200 mg of vanillin and, 2 mL of benzyl benzoate, 2 mL of anethole, and 2 mL of benzyl alcohol, respectively, were added to tubes E, F and G. There was complete solubilization of anetholetrithione when tubes C, D, E, F and G were stirred at room temperature. Anetholetrithione did not completely dissolve in tubes A and B even after heating at 60° C.

What is claimed is:

1. A composition comprising (i) an insoluble or a poorly-water soluble biologically active ingredient and (ii) at least one aromatic flavoring compound with a melting point higher than 20° C., as a solubilizing agent or crystallization inhibitor agent, wherein the at least one aromatic flavoring compound with a melting point higher than 20° C. is chosen from hydroxy- or alcoxy-substituted benzilic compounds or mixtures thereof.

2. The composition according to claim 1, wherein the flavoring compound with a melting point higher than 20° C. is chosen from vanillin, ethylvanillin, trans-anethole, cumarin, and mixtures thereof.

3. The composition according to claim 1, wherein the insoluble or poorly-water soluble biologically active ingredient is a pharmaceutical or an agrochemical ingredient.

4. The composition according to claim 3, wherein the insoluble or poorly-water soluble biologically active ingredient is a pharmaceutical ingredient.

5. The composition according to claim 4, wherein the pharmaceutical ingredient is chosen from modafinil, drospirenone, eplerenone, raloxifene and anetholtrithione.

6. The composition according to claim 5, wherein the pharmaceutical ingredient is anetholtrithione.

7. The composition according to claim 5, wherein the pharmaceutical ingredient is modafinil.

8. The composition according to claim 1, further comprising an excipient chosen from an aromatic organic solvent which is liquid at room temperature, a water soluble organic solvent, a nonionic surfactant, a water insoluble organic solvent, a semisolid organic compound, a liquid organic compound, a phospholipid, a pH regulator, and mixtures thereof.

9. The composition according to claim 8, wherein the excipient is an aromatic organic solvent chosen from benzilic alcohol and ethyl benzoate.

10. The composition according to claim 8, wherein the excipient is a water soluble organic solvent chosen from polyethyleneglycol 300, polyethyleneglycol 400, ethanol, propyleneglycol, glycerin, n-methylpyrrolidone, dimethylacetamide, and dimethyl sulfoxide.

11. The composition according to claim 8, wherein the excipient is a nonionic surfactant chosen from Cremofor EL, Cremofor RH 40, Cremofor RH 60, d-alpha-tocopherol, polyethyleneglycol succinate 1000, polysorbate 20, polysorbate 80, Solutol H15, sorbitane monooleate, poloxamer 407, Labrafil M-1944CS, Labrafil M-2125CS, Labrasol, Gellucire 44/14, Soften 767, and polyethylene glycol mono- and di-fat esters.

12. The composition according to claim 8, wherein the excipient is a water insoluble organic solvent chosen from linseed oil, corn oil, cottonseed oil, olive oil, peanut oil, mint oil, sunflower oil, soy oil, vegetable hydrogenated oil, and medium chain triglycerides of coconut oil.

13. The composition according to claim 8, wherein the excipient is a semi solid or a liquid organic compound chosen from bee's wax, oleic acid, and medium chain mono- and di-glycerides.

14. The composition according to claim 8, wherein the excipient is a phospholipid chosen from hydrogenated soy phosphatidylcholine, di-stearoyl-phosphatidylglycerol, L-α-di-myristoilphosphatidyl-choline, and L-α-di-myristoilphosphatidyl glycerol.

15. A pharmaceutical composition comprising (i) an insoluble or a poorly-water soluble pharmaceutically active ingredient; (ii) at least one aromatic flavoring compound with a melting point higher than 20° C., as a solubilizing agent or a crystallization inhibitor agent; and (iii) optionally further excipients, wherein the at least one aromatic flavoring compound with a melting point higher than 20° C. is chosen from benzilic compounds hydroxy- or alcoxy-substituted or mixtures thereof.

16. The pharmaceutical composition according to claim 15, wherein the aromatic flavoring compound with a melting point higher than 20° C. is chosen from vanillin, ethylvanillin, trans-anethole, cumarin, and mixtures thereof.

17. The pharmaceutical composition according to claim 15, wherein the active ingredient is chosen from modafinil, drospirenone, eplerenone, raloxifene, and anetholtrithione.

18. The pharmaceutical composition according to claim 17, wherein the active ingredient is anetholtrithione.

19. The pharmaceutical composition according to claim 17, wherein the active ingredient is modafinil.

20. The pharmaceutical composition according to claim 15, wherein the excipient is chosen from an aromatic organic solvent which is liquid at room temperature, a water soluble organic solvent, an nonionic surfactant, a water insoluble organic solvent, a semisolid organic compound, a liquid organic compound, a phospholipid, a pH regulator, and mixtures thereof.

21. The pharmaceutical composition according to claim 20, wherein the excipient is an aromatic organic solvent chosen from benzilic alcohol and ethyl benzoate.

22. The pharmaceutical composition according to claim 20, wherein the excipient is a water soluble organic solvent chosen from polyethyleneglycol 300, polyethyleneglycol 400, ethanol, propyleneglycol, glycerin, n-methylpyrrolidone, dimethylacetamide, and dimethyl sulfoxide.

23. The pharmaceutical composition according to claim 20, wherein the excipient is a nonionic surfactant chosen from Cremofor EL, Cremofor RH 40, Cremofor RH 60, d-alpha-tocopherol, polyethyleneglycol succinate 1000, polysorbate 20, polysorbate 80, Solutol H15, sorbitane monooleate, poloxamer 407, Labrafil M-1944CS, Labrafil M-2125CS, Labrasol, Gellucire 44/14, Soften 767, and polyethylene glycol mono- and di-fat esters.

24. The pharmaceutical composition according to claim 20, wherein the excipient is a water insoluble organic solvent chosen from linseed oil, corn oil, cottonseed oil, olive oil, peanut oil, mint oil, sunflower oil, soy oil, vegetable hydrogenated oil, and medium chain triglycerides of coconut oil.

25. The pharmaceutical composition according to claim 20, wherein the excipient is a semi solid or a liquid organic compound chosen from bee's wax, oleic acid, and medium chain mono- and di-glycerides.

26. The pharmaceutical composition according to claim 20, wherein the excipient is a phospholipid chosen from hydrogenated soy phosphatidylcholine, di-stearoyl-phosphatidylglycerol, L-α-di-myristoilphosphatidyl-choline, and L-α-di-myristoilphosphatidyl glycerol.

27. The pharmaceutical composition according to claim 15, wherein the at least one aromatic flavoring compound is present in an amount of more than one tenth of the mass of the active ingredient.

28. The pharmaceutical composition according to claim 15, wherein the at least one aromatic flavoring compound is present in an amount higher than 10% by wt relative to the other excipients.

29. The pharmaceutical composition according to claim 15, wherein the at least one aromatic flavoring compound is substantially free of impurities.

30. The pharmaceutical composition according to claim 15, wherein said composition is a self-emulsifying or micro- or nanoemulsified composition.

31. A process for solubilizing or inhibiting crystallization of at least one insoluble or poorly-water soluble biologically active ingredient in a pharmaceutical or agrochemical composition, the process comprising bringing at least one aromatic flavoring compound having a melting point of higher than 20° C. into contact with at the least one active ingredient.
